(19) United States
(12) Patent Application Publication
(10) Pub. No.: US 2023/0256157 A1
(43) Pub. Date: Aug. 17, 2023

(54) Ear Canal Cleaning and Cerumen Sample Collecting Apparatus for cleaning and removing cerumen

(71) Applicant: Ronald Dennis Smith, JR., Portage, PA (US)

(72) Inventor: Ronald Dennis Smith, JR., Portage, PA (US)

(21) Appl. No.: 18/004,919

(22) PCT Filed: Nov. 5, 2020
(86) PCT No.: PCT/US2020/070750

§ 371 Date: Jan. 10, 2023

(51) Int. Cl. A61M 3/02

(52) U.S. Cl. CPC A61M 3/0262; A61M 2210/0662; A61M 2205/50

(57) ABSTRACT

The Ear Canal Cleaning and Cerumen Sample Collecting Apparatus is either a manual pulsing or a recirculating fluid transfer syringe used to clean and remove cerumen from the ear as well as collect a sample of cerumen for testing. The Ear Canal Cleaning and Cerumen Sample Collecting Apparatus can be fully automated, and cordless. The Ear Canal Cleaning and Cerumen Sample Collecting Apparatus can be arranged into any ergonomic shape or configured as a single hand, double hand, palm held, or headphone-like device to clean, remove, and sample cerumen.

### TECHNICAL FIELD OF THE INVENTION

This invention relates generally to an Ear Canal Cleaning and Cerumen Sample Collecting Apparatus in the form of a fluid flushing system fitting into an ear canal for the purpose of cleaning, removing, and sampling cerumen. This invention relates generally to cerumen cleaning and sampling devices. This invention relates specifically to instruments inserted into the ear for the purpose of cleaning and removing cerumen from the ear canal. This invention relates specifically to fluid based cerumen cleaning and sampling instruments and devices.

### BACKGROUND OF THE INVENTION

Cerumen, ear wax, is a naturally occurring waxy and oily organic compound produced by the body to coat and protect the ear canal and prevent detritus from getting in contact with the ear drum. The body continually produces cerumen. Cerumen is naturally ejected by the body by the usual motions of talking and chewing. At times, and in the elderly and infirm, cerumen can build up and require cleaning and removal. Further, recent advances have led to many available tests for disease states and conditions that can be reliably conducted with cerumen.

In searching for a non-intrusive, easy to use, non-messy, home device for earwax removal that is capable of collecting a sample for testing, there are various mechanical, and vacuum based devices available today that can perform cerumen removal, some even that can collect a sample. None are non-messy and offer a convenient, no-touch cerumen collection means. When focused on home user, reliable cerumen sample collection, and the possibility of a clinical diagnostic use of a cerumen sample that is easy and convenient to collect, there is no device available today that does not require complex, potentially sample contaminating handling.

Recently published research indicates a strong need for a good reliable home use user operated cerumen cleaning and removal device that can reliably and easily collect a cerumen sample for testing. Further, compacted earwax is a persistent medical condition, especially among the elderly, that is not adequately addressed by any existing home healthcare earwax removal products, either for self-care or those used in assisted care by non-medical personnel.

In light of the foregoing prior art, there is a need for a better cerumen cleaning and removal device that can easily and simply collect a sample that does not require complex handling and processing.

### BRIEF SUMMARY OF THE INVENTION

According to a first aspect of the invention, there is an Ear Canal Cleaning and Cerumen Sample Collecting Apparatus for cleaning and removing cerumen from an ear comprising a fluid inside a syringe having an open end having an one-way in-flow valve positioned in the center occupying up to fifty percent of the area of said open end, and a closed end wherein a plunger passes through said closed end said plunger comprising a plunger head, a plunger arm, an internal tension spring around said plunger arm connectively attached to and positioned between said plunger head and said closed end; and a plunger handle enabling cleaning and removing cerumen from said ear by positioning said open end so as to cover an external opening into an ear canal by producing an outward pulse of said fluid exiting said syringe as said plunger handle is pushed up to half-way into said syringe thereby compressing said tension spring and thereby storing a spring tension force and an inward pulse of said fluid entering said syringe as said internal tension spring releases said stored spring tension force.

According to a second aspect of the invention there is a Ear Canal Cleaning and Cerumen Sample Collecting Apparatus for cleaning and removing cerumen from an ear comprising a fluid inside a syringe having an open end and a closed end having a plunger head having a plurality of one-way flow valves and a plurality of plunger arms connectively attached to said plunger head and passing through said closed end said plunger arms comprise a plunger head connection, a plunger arm, an internal tension spring around said plunger arm connectively attached to and positioned between said plunger head and said closed end, and a plunger handle, wherein an internal tube is positioned down a center line of said syringe so as to occupy up to fifty percent of the internal volume of said syringe positioned from said open end to said closed end wherein said internal tube comprises at least one one-way in-flow valve positioned in said internal tube at said open end and a plurality of holes passing through said internal tube positioned at said closed end enabling cleaning and removing cerumen from said ear by positioning said open end so as to cover an external opening into an ear canal by producing an outward pulse of said fluid exiting said syringe as said plunger handle is pushed up to half-way into said syringe thereby compressing said tension spring and thereby storing a spring tension force and an inward pulse of said fluid entering said syringe as said internal tension spring releases said stored spring tension force.

According to a third aspect of the invention there is an Ear Canal Cleaning and Cerumen Sample Collecting Apparatus for cleaning and removing cerumen further comprising an ear canal connection plug connectively attached to said open end enabling cleaning and removing of cerumen from said ear.

According to a fourth aspect of the invention there is an Ear Canal Cleaning and Cerumen Sample Collecting Apparatus for cleaning, removing, and sampling cerumen further comprising a cerumen sampling collar connectively attached to said open end enabling cleaning, removing, and sampling of cerumen from said ear.

According to a fifth aspect of the invention there is an Ear Canal Cleaning and Cerumen Sample Collecting Apparatus for cleaning, removing, and sampling cerumen further comprising a cerumen sampling collar connectively attached to said open end and an ear canal connection plug connectively attached to said sampling collar enabling cleaning, removing, and sampling of cerumen from said ear.

According to a sixth aspect of the invention there is an Ear Canal Cleaning and Cerumen Sample Collecting Apparatus for cleaning, removing, and sampling cerumen further comprising a handheld ergonomic container housing said apparatus having a trigger connectively attached to a digital controller supplied electric power by a battery, a battery charging port connectively attached to said battery, an actuator connectively attached to said plunger handle and said digital controller and controlled by said digital controller.

An advantage of the present invention is cerumen can be self flushed and removed as well as sampled for testing.

The invention will now be described, by way of example only, with reference to the accompanying drawings in which:

### BRIEF DESCRIPTION OF THE DRAWINGS

FIG. 1 is a cross section view of a gun shaped embodiment according to the invention;

FIG. 2 is a perspective view showing the three main components of a recirculating fluid system embodiment shown separated apart according to the invention;

FIG. 3A is a side view showing the three main components of a recirculating fluid system embodiment connected together according to the invention;

FIG. 3B is a half-section side view showing the three main components of a recirculating fluid system embodiment connected together according to the invention;

FIG. 4A is a side view showing the three main components of a pulsing fluid system embodiment shown separated apart according to the invention; and

FIG. 4B is a half-section side view showing the three main components of a pulsing fluid system embodiment shown separated apart according to the invention.

### DETAILED DESCRIPTION

The detailed embodiments of the present invention are disclosed herein. The disclosed embodiments are merely exemplary of the invention, which may be embodied in various forms. The details disclosed herein are not to be interpreted as limiting, but merely as the basis for the claims and as a basis for teaching one skilled in the art how to make and use the invention.

References in the specification to “one embodiment,” “an embodiment,” “an example embodiment,” etcetera, indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

Furthermore, it should be understood that spatial descriptions (e.g., “above,” “below,” “up,” “left,” “right,” “down,” “top,” “bottom,” “vertical,” “horizontal,” etc.) used herein are for purposes of illustration only, and that practical implementations of the structures described herein can be spatially arranged in any orientation or manner.

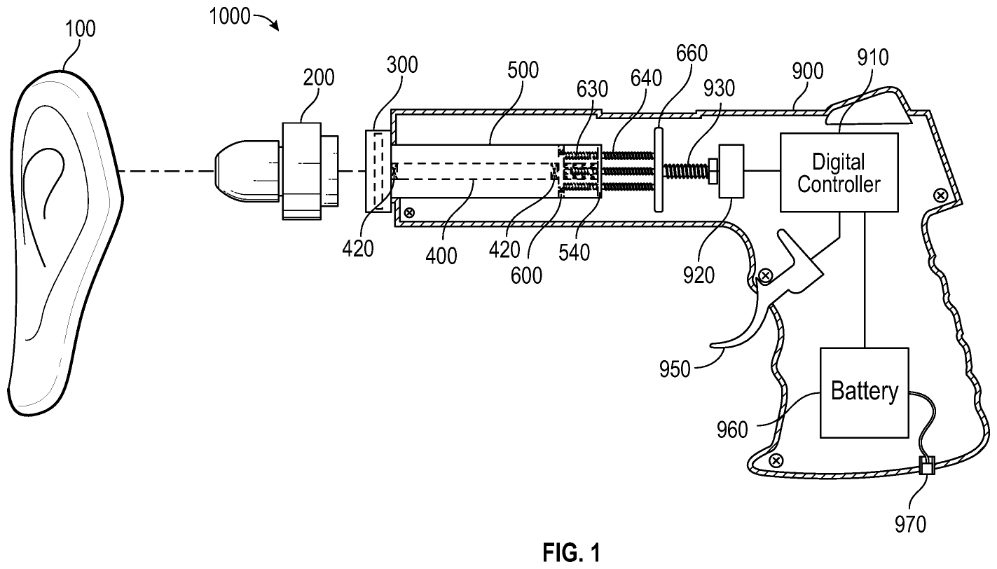

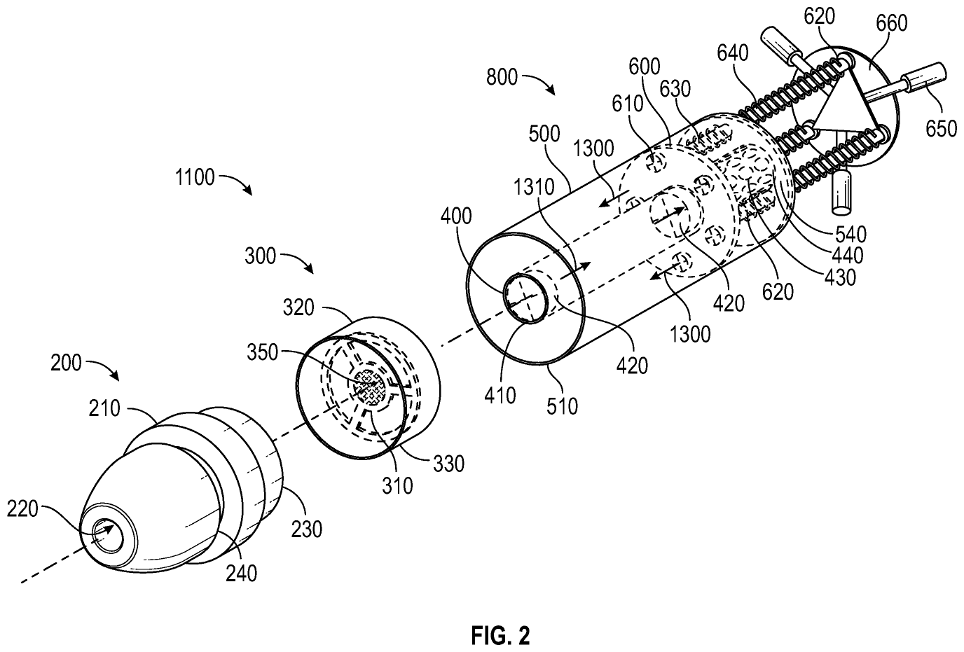

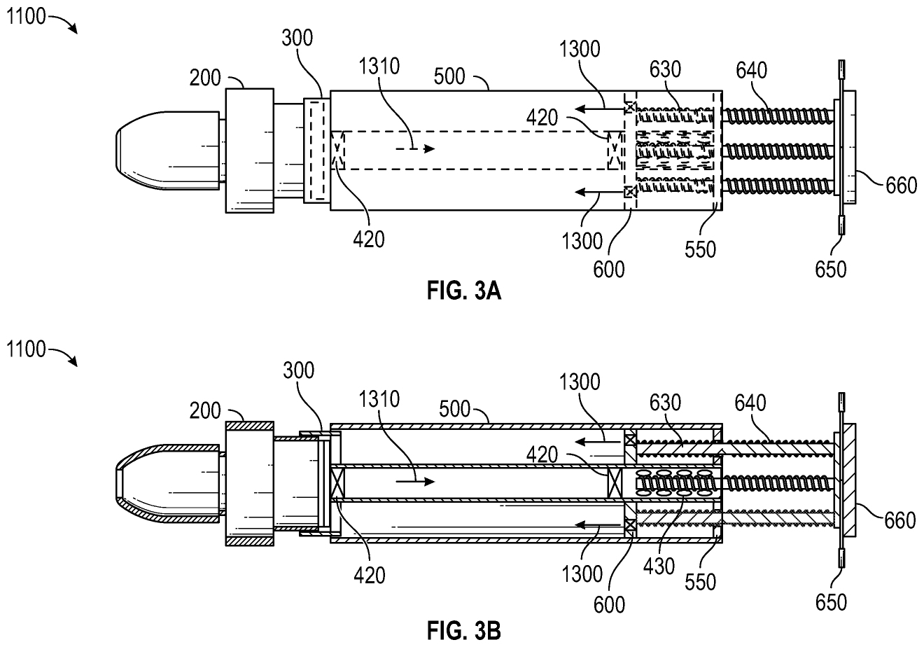

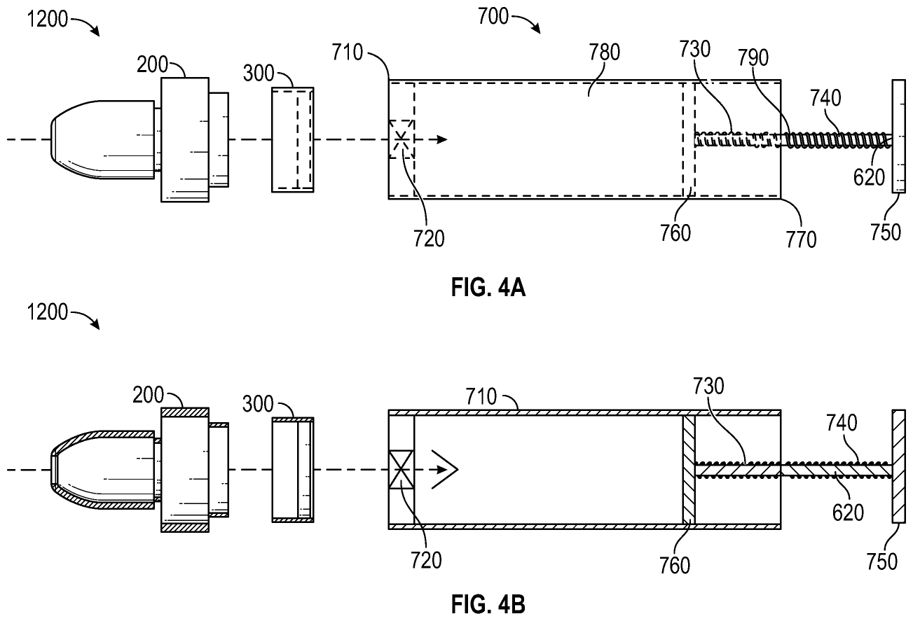

Referring to the Figures, there is shown in FIG. 1, Element 1000, which is gun shaped embodiment of the Ear Canal Cleaning and Cerumen Sample Collecting Apparatus. There is shown in FIGS. 2, 3A, and 3B, Element 1100 ,which is recirculating fluid embodiment of the Ear Canal Cleaning and Cerumen Sample Collecting Apparatus. There is shown in FIGS. 4A and 4B, Element 1200, which is pulsing fluid embodiment of the Ear Canal Cleaning and Cerumen Sample Collecting Apparatus. Element 1300, is the fluid flow direction out the apparatus and into an ear canal. Element 1310, is the fluid flow direction out of an ear canal and into the apparatus.

Index of Labelled Elements in Figures (in numeric order):

Element 100 is an ear.

Element 200 is an ear canal connection plug.

Element 210 is an ear canal seal.

Element 220 is an opening for fluid in said ear canal connection plug (200).

Element 230 is an ear canal connection plug connection interface.

Element 240 is an ear canal opening seal.

Element 300 is a cerumen catchment collar.

Element 310 is a cerumen catchment screen frame.

Element 320 is a rearward cerumen catchment connection interface.

Element 330 is a forward cerumen catchment connection interface.

Element 350 is a cerumen catchment screen.

Element 400 is an internal tube.

Element 410 is an open end on an internal tube (400).

Element 420 is an one-way in-flow valve.

Element 430 is a hole in the closed end on an internal tube (400).

Element 440 is a closed end on an internal tube (400).

Element 500 is a recirculating syringe for a recirculating embodiment.

Element 510 is an open end on a recirculating syringe (500).

Element 540 is a closed end on a recirculating syringe (500).

Element 600 is a valved plunger head.

Element 610 is an one-way out-flow valve in a valved plunger head (600).

Element 620 is a plunger arm.

Element 630 is an internal tension spring coil.

Element 640 is an external tension spring coil.

Element 650 is a plunger handle.

Element 660 is a plunger push plate.

Element 700 is a pulsing syringe for pulsing embodiment.

Element 710 is an open end of a syringe (700).

Element 720 is a suspended one-way in-flow valve.

Element 730 is an internally positioned tension spring coil.

Element 740 is an external positioned tension spring coil.

Element 750 is plunger arm push plate.

Element 760 is a solid plunger head.

Element 770 is a closed end of a pulsing syringe (700).

Element 780 is a fluid inside a pulsing syringe (700).

Element 790 is a plunger arm, similar to plunger arm (620).

Element 800 is a recirculating fluid type syringe.

Element 900 is an handheld ergonomic container housing.

Element 910 is a digital controller.

Element 920 is an electric drive motor.

Element 930 is a reversible screw drive.

Element 950 is a trigger switch.

Element 960 is a battery.

Element 970 is a battery charging port.

A syringe is a simple reciprocating pump consisting of a plunger that fits tightly within a cylindrical tube. The plunger can be pushed and pulled along the inside of the tube, allowing the syringe to take in and expel a fluid (780)/liquid or solution through the open end of the tube.

In a preferred embodiment of the present invention, as in FIGS. 4A and 4B, there is an Ear Canal Cleaning and Cerumen Sample Collecting Apparatus for cleaning and removing cerumen from an ear comprising a fluid (780) inside a pulsing syringe (700) having an open end (710) having a suspended one-way in-flow valve (720) positioned in the center occupying up to fifty percent of the area of said open end (710), and a closed end (770) wherein a solid head plunger (760) passes through said closed end (770); said plunger comprising a solid plunger head (760), a plunger arm (620), an internal tension spring (730) around said plunger arm (620) connectively attached to and positioned between said solid plunger head (760) and said closed end (770), and a plunger arm push plate (750) enabling cleaning and removing cerumen from said ear (100) by positioning said open end (710) so as to cover an external opening into an ear canal by producing an outward pulse of said fluid (780) exiting said pulsing syringe (700) as said plunger arm push plate (750) is pushed up to half-way into said pulsing syringe (700) thereby compressing said internal tension spring (730) and thereby storing a spring tension force and an inward pulse of said fluid (780) entering said syringe as said internal tension spring (730) releases said stored spring tension force.

In the pulsing syringe (700) embodiment the fluid (780) used for cleaning is preferably water. Any suitable water, solution, or tincture may be used as the fluid (780). The fluid (780) is pulsed a plurality of times to fill and empty the ear canal with fluid thereby using the fluid (780) to flush, clean, remove, and wash out cerumen build up within an ear canal. The plunger arm push plate (750) is depressed/pushed causing the solid plunger head (760) to advance into the syringe body approximately fifty percent down the length of the pulsing syringe (700). The pushing action causes the internal tension spring (730) to compress, and this compression imparts stored force in the internal tension spring (730). After the plunger head (760) is advanced, the plunger head is returned to its original (normal, or resting) position by the force stored in the internal tension spring (730) during pushing. After the internal tension spring (730) returns the plunger head (760) to its resting position, the plunger head (760) depression is repeated. This cycle continues for one to one hundred strokes, or more if needed for cleaning, removing, and optionally sampling cerumen depending on the embodiment of the invention.

In a preferred embodiment of the present invention, as in FIGS. 2, 3A, and 3B there is an Ear Canal Cleaning and Cerumen Sample Collecting Apparatus for cleaning and removing cerumen from an ear (100) comprising a fluid (780) inside a recirculating syringe (500) having an open end (510) and a closed end (540) having a valved plunger head (600) having a plurality of one-way in-flow valves (420) and a plurality of plunger arms (620) connectively attached to said valved plunger head (600) and passing through said closed end (540) said plunger arms (620) comprise a valved plunger head (600) connection, a plunger arm (620), an internal tension spring coil (630) around said plunger arm (620) connectively attached to and positioned between said valved plunger head (600) and said closed end (540), and a plunger handle (650), wherein an internal tube (400) is positioned down a center line of said recirculating syringe (500) so as to occupy up to fifty percent of the internal volume of said recirculating syringe (500) positioned from said open end (510) to said closed end (540) wherein said internal tube (400) comprises at least one one-way in-flow valve (420) positioned in said internal tube (400) at said open end (410, 510) and a plurality of holes (430) passing through said internal tube (400) positioned at said closed end (510) enabling cleaning and removing cerumen from said ear (100) by positioning said open end (510) so as to cover an external opening into an ear canal by producing an outward pulse of said fluid (780) exiting said recirculating syringe (500) as said plunger handle (650) is pushed up to half-way into said recirculating syringe (500) thereby compressing said internal tension spring coil (630) and thereby storing a spring tension force and an inward pulse of said fluid (780) entering said syringe as said internal tension spring coil (630) releases said stored spring tension force.

In the recirculating fluid type syringe (800) embodiment the fluid (780) used for cleaning is preferably water. However, any suitable liquid, solution, or tincture may be used as the fluid (780).

In the recirculating fluid type syringe (800) embodiment the fluid (780) is recirculated a plurality of times to fill and empty the ear canal with fluid thereby using the fluid (780) to flush, clean, remove, and wash out cerumen build up within an ear canal. The valved plunger head (600) is advanced into the syringe body approximately fifty percent down the length of the recirculating syringe (500) sending fluid into an ear canal out of the recirculating fluid type syringe (800) at the open end (510) of the recirculating syringe (500). This advancing action causes the internal tension spring coil (630) to compress, and this compression imparts stored force in the internal tension spring coil (630). After the valved plunger head (600) is advanced, it is returned to its original (normal, or resting) position by the force stored in the internal tension spring coil (630) during pushing. After the internal tension spring (630) returns the plunger head (600) to its resting position, the plunger head (600) is advanced and the cycle repeated. This cycle continues for one to one hundred strokes, or more if needed, cleaning, removing, and optionally sampling cerumen depending on the embodiment of the invention. There is no real limit to the number of cleaning strokes that can be applied with regular rinsing and cleaning of the recirculating fluid type syringe (800) and refilling with new/clean fluid (780).

In the recirculating fluid type syringe (800), as the valved plunger head (600) is advanced, fluid exits the recirculating syringe (500) at its open end (510), and at the same time, can enter the recirculating syringe (500) through the internal tube (400) at its open end (410) This enables a continuous flow of fluid into an ear canal, enabling continuous rinsing because the fluid continues to exit and enter the recirculating fluid type syringe (800) with each stroke cycle.

The catchment collar (300) is used when cleaning, removing, and sampling cerumen. The catchment collar (300) is comprised of a cerumen catchment screen frame (310), a rearward cerumen catchment connection interface (320), a forward cerumen catchment connection interface (330) and a removable a cerumen catchment screen (350).

The ear (100) connection device is an ear canal connection plug (200) comprised of an ear canal seal (210), a fluid opening (220), an ear canal connection plug connection interface (230), and an ear canal opening seal (240).

The Ear Canal Cleaning and Cerumen Sample Collecting Apparatus may alternatively be further comprising an ear canal connection plug (200) connectively attached to said open end (510, 710) enabling cleaning and removing of cerumen from said ear (100).

The Ear Canal Cleaning and Cerumen Sample Collecting Apparatus may alternatively be further comprising a cerumen catchment (sampling) collar (300) connectively attached to said open end (510, 710) enabling cleaning, removing, and sampling of cerumen from said ear (100).

The Ear Canal Cleaning and Cerumen Sample Collecting Apparatus may alternatively be further comprising both an ear canal connection plug (200) connectively attached to said open end (510, 710), and a cerumen catchment (sampling) collar (300) connectively attached to said open end (510, 710) for enabling cleaning, removing, and sampling of cerumen from said ear (100).

The Ear Canal Cleaning and Cerumen Sample Collecting Apparatus may alternatively be further comprising a handheld ergonomic container housing (900); said apparatus having a trigger switch (950) connectively attached to a digital controller (910) supplied electric power by a battery (960), a battery charging port (970) connectively attached to said battery (960), an actuator in the form of an electric drive motor (920) connectively attached to a plunger push plate (660) and said digital controller (910) and controlled by said digital controller (910).

In an alternate embodiment, the Ear Canal Cleaning and Cerumen Sample Collecting Apparatus is comprised of two automated, or manual, individual Ear Canal Cleaning and Cerumen Sample Collecting Apparatuses arranged as a set of headphones.

The invention has been described by way of examples only. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the claims.

Although the invention has been explained in relation to various embodiments, it is to be understood that many other possible modifications and variations can be made without departing from the spirit and scope of the invention.

1. An Ear Canal Cleaning and Cerumen Sample Collecting Apparatus for cleaning and removing cerumen from an ear comprising a fluid inside a syringe having an open end having an one-way in-flow valve positioned in the center occupying up to fifty percent of the area of said open end, and a closed end wherein a plunger passes through said closed end, said plunger comprising a plunger head, a plunger arm, an internal tension spring around said plunger arm connectively attached to and positioned between said plunger head and said closed end, and a plunger handle enabling cleaning and removing cerumen from said ear by positioning said open end so as to cover an external opening into an ear canal by producing an outward pulse of said fluid exiting said syringe as said plunger handle is pushed up to half-way into said syringe thereby compressing said tension spring and thereby storing a spring tension force and an inward pulse of said fluid entering said syringe as said internal tension spring releases said stored spring tension force.

2. The apparatus of claim 1 for cleaning and removing cerumen further comprising an ear canal connection plug connectively attached to said open end enabling cleaning and removing of cerumen from said ear by positioning said ear canal connection plug so as to cover an external opening into an ear canal by producing an outward pulse of said fluid exiting said syringe as said plunger handle is pushed up to half-way into said syringe thereby compressing said tension spring and thereby storing a spring tension force and an inward pulse of said fluid entering said syringe as said internal tension spring releases said stored spring tension force.

3. The apparatus of claim 1 for cleaning, removing, and sampling cerumen further comprising a cerumen sampling collar connectively attached to said open end enabling cleaning, removing, and sampling of cerumen from said ear by positioning said cerumen sampling collar so as to cover an external opening into an ear canal by producing an outward pulse of said fluid exiting said syringe as said plunger handle is pushed up to half-way into said syringe thereby compressing said tension spring and thereby storing a spring tension force and an inward pulse of said fluid entering said syringe as said internal tension spring releases said stored spring tension force.

4. The apparatus of claim 1 for cleaning, removing, and sampling cerumen further comprising a cerumen sampling collar connectively attached to said open end and an ear canal connection plug connectively attached to said sampling collar enabling cleaning, removing, and sampling of cerumen from said ear by positioning said cerumen sampling collar so as to cover an external opening into an ear canal by producing an outward pulse of said fluid exiting said syringe as said plunger handle is pushed up to half-way into said syringe thereby compressing said tension spring and thereby storing a spring tension force and an inward pulse of said fluid entering said syringe as said internal tension spring releases said stored spring tension force.

5. The apparatus of claim 1 for cleaning and removing cerumen further comprising a handheld ergonomic container housing said apparatus having a trigger connectively attached to a digital controller supplied electric power by a battery, a battery charging port connectively attached to said battery, an actuator connectively attached to said plunger handle and said digital controller and controlled by said digital controller.

6. The apparatus of claim 1 for cleaning and removing cerumen further comprising a handheld ergonomic container housing said apparatus having a trigger connectively attached to a digital controller supplied electric power by a battery, a battery charging port connectively attached to said battery, an actuator connectively attached to said plunger handle and said digital controller and controlled by said digital controller, and further comprising an ear canal connection plug connectively attached to said open end enabling cleaning and removing of cerumen from said ear by positioning said ear canal connection plug so as to cover an external opening into an ear canal by producing an outward pulse of said fluid exiting said syringe as said plunger handle is pushed up to half-way into said syringe thereby compressing said tension spring and thereby storing a spring tension force and an inward pulse of said fluid entering said syringe as said internal tension spring releases said stored spring tension force.

7. The apparatus of claim 1 for cleaning, removing, and sampling cerumen further comprising a handheld ergonomic container housing said apparatus having a trigger connectively attached to a digital controller supplied electric power by a battery, a battery charging port connectively attached to said battery, an actuator connectively attached to said plunger handle and said digital controller and controlled by said digital controller, and further comprising a cerumen sampling collar connectively attached to said open end enabling cleaning, removing, and sampling of cerumen from said ear by positioning said cerumen sampling collar so as to cover an external opening into an ear canal by producing an outward pulse of said fluid exiting said syringe as said plunger handle is pushed up to half-way into said syringe thereby compressing said tension spring and thereby storing a spring tension force and an inward pulse of said fluid entering said syringe as said internal tension spring releases said stored spring tension force.

8. The apparatus of claim 1 for cleaning, removing, and sampling cerumen further comprising a handheld ergonomic container housing said apparatus having a trigger connectively attached to a digital controller supplied electric power by a battery, a battery charging port connectively attached to said battery, an actuator connectively attached to said plunger handle and said digital controller and controlled by said digital controller, and further comprising a cerumen sampling collar connectively attached to said open end and an ear canal connection plug connectively attached to said sampling collar enabling cleaning, removing, and sampling of cerumen from said ear by positioning said cerumen sampling collar so as to cover an external opening into an ear canal by producing an outward pulse of said fluid exiting said syringe as said plunger handle is pushed up to half-way into said syringe thereby compressing said tension spring and thereby storing a spring tension force and an inward pulse of said fluid entering said syringe as said internal tension spring releases said stored spring tension force.

9. An Ear Canal Cleaning and Cerumen Sample Collecting Apparatus for cleaning and removing cerumen from an ear comprising a fluid inside a syringe having an open end and a closed end having a plunger head having a plurality of one-way flow valves and a plurality of plunger arms connectively attached to said plunger head and passing through said closed end, said plunger arms comprise a plunger head connection, a plunger arm, an internal tension spring around said plunger arm connectively attached to and positioned between said plunger head and said closed end, and a plunger handle, wherein an internal tube is positioned down a center line of said syringe so as to occupy up to fifty percent of the internal volume of said syringe positioned from said open end to said closed end wherein said internal tube comprises at least one one-way inflow valve positioned in said internal tube at said open end and a plurality of holes passing through said internal tube positioned at said closed end enabling cleaning and removing cerumen from said ear by positioning said open end so as to cover an external opening into an ear canal by producing an outward pulse of said fluid exiting said syringe as said plunger handle is pushed up to half-way into said syringe thereby compressing said tension spring and thereby storing a spring tension force and an inward pulse of said fluid entering said syringe as said internal tension spring releases said stored spring tension force.

10. The apparatus of claim 9 for cleaning and removing cerumen further comprising an ear canal connection plug connectively attached to said open end enabling cleaning and removing of cerumen from said ear by positioning said ear canal connection plug so as to cover an external opening into an ear canal by producing an outward pulse of said fluid exiting said syringe as said plunger handle is pushed up to half-way into said syringe thereby compressing said tension spring and thereby storing a spring tension force and an inward pulse of said fluid entering said syringe as said internal tension spring releases said stored spring tension force.

11. The apparatus of claim 9 for cleaning, removing, and sampling cerumen further comprising a cerumen sampling collar connectively attached to said open end enabling cleaning, removing, and sampling of cerumen from said ear by positioning said cerumen sampling collar so as to cover an external opening into an ear canal by producing an outward pulse of said fluid exiting said syringe as said plunger handle is pushed up to half-way into said syringe thereby compressing said tension spring and thereby storing a spring tension force and an inward pulse of said fluid entering said syringe as said internal tension spring releases said stored spring tension force.

12. The apparatus of claim 9 for cleaning, removing, and sampling cerumen further comprising a cerumen sampling collar connectively attached to said open end and an ear canal connection plug connectively attached to said sampling collar enabling cleaning, removing, and sampling of cerumen from said ear by positioning said cerumen sampling collar so as to cover an external opening into an ear canal by producing an outward pulse of said fluid exiting said syringe as said plunger handle is pushed up to half-way into said syringe thereby compressing said tension spring and thereby storing a spring tension force and an inward pulse of said fluid entering said syringe as said internal tension spring releases said stored spring tension force.

13. The apparatus of claim 9 for cleaning and removing cerumen further comprising a handheld ergonomic container housing said apparatus having a trigger connectively attached to a digital controller supplied electric power by a battery, a battery charging port connectively attached to said battery, an actuator connectively attached to said plunger handle and said digital controller and controlled by said digital controller.

14. The apparatus of claim 9 for cleaning and removing cerumen further comprising a handheld ergonomic container housing said apparatus having a trigger connectively attached to a digital controller supplied electric power by a battery, a battery charging port connectively attached to said battery, an actuator connectively attached to said plunger handle and said digital controller and controlled by said digital controller, and further comprising an ear canal connection plug connectively attached to said open end enabling cleaning and removing of cerumen from said ear by positioning said ear canal connection plug so as to cover an external opening into an ear canal by producing an outward pulse of said fluid exiting said syringe as said plunger handle is pushed up to half-way into said syringe thereby compressing said tension spring and thereby storing a spring tension force and an inward pulse of said fluid entering said syringe as said internal tension spring releases said stored spring tension force.

15. The apparatus of claim 9 for cleaning, removing, and sampling cerumen further comprising a handheld ergonomic container housing said apparatus having a trigger connectively attached to a digital controller supplied electric power by a battery, a battery charging port connectively attached to said battery, an actuator connectively attached to said plunger handle and said digital controller and controlled by said digital controller, and further comprising a cerumen sampling collar connectively attached to said open end enabling cleaning, removing, and sampling of cerumen from said ear by positioning said cerumen sampling collar so as to cover an external opening into an ear canal by producing an outward pulse of said fluid exiting said syringe as said plunger handle is pushed up to half-way into said syringe thereby compressing said tension spring and thereby storing a spring tension force and an inward pulse of said fluid entering said syringe as said internal tension spring releases said stored spring tension force.

16. The apparatus of claim 9 for cleaning, removing, and sampling cerumen further comprising a handheld ergonomic container housing said apparatus having a trigger connectively attached to a digital controller supplied electric power by a battery, a battery charging port connectively attached to said battery, an actuator connectively attached to said plunger handle and said digital controller and controlled by said digital controller, and further comprising a cerumen sampling collar connectively attached to said open end and an ear canal connection plug connectively attached to said sampling collar enabling cleaning, removing, and sampling of cerumen from said ear by positioning said cerumen sampling collar so as to cover an external opening into an ear canal by producing an outward pulse of said fluid exiting said syringe as said plunger handle is pushed up to half-way into said syringe thereby compressing said tension spring and thereby storing a spring tension force and an inward pulse of said fluid entering said syringe as said internal tension spring releases said stored spring tension force.
